(19) United States
(12) Patent Application Publication
(10) Pub. No.: US 2023/0211145 A1
(43) Pub. Date: Jul. 6, 2023

(54) CIRCULATORY ASSIST DEVICES, AND RELATED METHODS

(71) Applicant: Second Heart Assist, Inc., Salt Lake City, UT (US)

(72) Inventor: Alex Richardson, Thousand Oaks, CA (US)

(21) Appl. No.: 18/150,057

(22) Filed: Jan. 4, 2023

Related U.S. Application Data
(60) Provisional application No. 63/296,780, filed on Jan. 5, 2022.

(51) Int. Cl. A61M 60/81; A61M 60/416; A61M 60/216; A61M 60/827; A61M 60/825; A61M 60/13; A61M 60/873

(52) U.S. Cl. CPC A61M 60/81; A61M 60/13; A61M 60/216; A61M 60/416; A61M 60/825; A61M 60/827; A61M 60/873

(57) ABSTRACT

A coupling for a circulatory assist device includes a first coupler and a second coupler. The first coupler including a first inner portion configured to be secured to a first shaft, a first body extending from the inner portion, and first magnets joined to the body. The second coupler offset from and separate from the first coupler with a gap therebetween. The second coupler including a second inner portion configured to be secured to a second shaft, a second body extending from the second inner portion, and second magnets joined to the body. The second magnets magnetically coupled to the first magnets and configured to transfer a torque applied to one of the first shaft and the second shaft to an other of the first shaft and the second shaft.

### CROSS REFERENCE TO RELATED APPLICATION

This application claims the benefit under 35 U.S.C. §119(e) of U.S. Provisional Patent Application Serial No. 63/296,780, entitled “CIRCULATORY ASSIST DEVICES, AND RELATED METHODS,” filed Jan. 5, 2022, the disclosure of which is hereby incorporated herein in its entirety by this reference.

### TECHNICAL FIELD

Embodiments of the application relate generally to medical devices, and in particular, to circulatory assist devices and associated methods for assisting a subject’s heart to circulate blood.

### BACKGROUND

Circulatory assist devices, such as pumps, may be used to assist a subject’s heart to pump blood in order to address conditions such as heart disease.

U.S. 2021/0077687 A1 to Leonhardt (Published Mar. 18, 2021), the contents of which are incorporated herein by this reference, relates to a circulatory support platform utilizing an aortic stent pump, comprising a stent cage enabling open flow and an impeller within the stent cage. The circulatory support platform may facilitate blood circulation and pulsatility. The circulatory support platform may include shape memory materials to adjust the shape and size of the impeller blades. Additionally, the circulatory support platform may be wirelessly operated.

U.S. Pat. 8,617,239 to Reitan (Dec. 31, 2013), the contents of which are incorporated herein by this reference, relates to a catheter pump to be positioned in the ascending aorta near the aortic valve of a human being, comprising an elongated sleeve with a drive cable extending through the sleeve and connectable at its proximal end to an external drive source and a drive rotor near the distal end of the drive cable mounted on a drive shaft being connected with the drive cable. The drive rotor consists of a propeller enclosed in a cage and the propeller and the cage are foldable from an insertion position close to the drive shaft to an expanded working position, which are characterized by means for anchoring the drive rotor in the ascending aorta near the aortic valve after insertion. Also described is a method to position the pump of a catheter pump in the ascending aorta just above the aortic valve.

The above-described background relating to circulatory assist devices is merely intended to provide a contextual overview of some current issues and is not intended to be exhaustive. Other contextual information may become apparent to those of ordinary skill in the art upon review of the following description, which includes example embodiments.

### BRIEF SUMMARY

Embodiments of the disclosure include a coupling for a circulatory assist device, a circulatory assist device, and related methods.

In one illustrative embodiment, the disclosure provides a coupling for a circulatory assist device. The coupling includes a first coupler and a second coupler. The first coupler includes a first inner portion configured to be secured to a first shaft, a first body extending from the inner portion, and first magnets joined to the body. The second coupler offset from and separate from the first coupler with a gap therebetween. The second coupler includes a second inner portion configured to be secured to a second shaft, a second body extending from the second inner portion, and second magnets joined to the body. The second magnets are magnetically coupled to the first magnets and configured to transfer a torque applied to one of the first shaft and the second shaft to an other of the first shaft and the second shaft

In another illustrative embodiment, the disclosure provides a circulatory assist device. The circulatory assist device includes a motor, a driveshaft, a seal, an impeller, and a coupling. The motor is positioned within a housing. The driveshaft extends from the motor within the housing. The seal forms a sealed compartment with the housing. The compartment confines the motor and the driveshaft therein. The impeller includes an impeller shaft. The impeller shaft is substantially axially aligned with the driveshaft and offset from the driveshaft with the seal extending therebetween. The coupling is configured to magnetically couple the driveshaft to the impeller shaft and to transfer torque from the motor to the impeller across the seal.

In a further illustrative embodiment, the disclosure provides a method of operating a circulatory assist device. The method includes rotating a driveshaft with a motor positioned within a housing, the driveshaft extending from the motor within the housing. The method also includes transferring a torque from the motor to an impeller across a seal via a coupling, the seal forming a sealed compartment with the housing and the compartment confining the motor and the driveshaft therein. The impeller includes an impeller shaft. The impeller shaft substantially axially aligns with the driveshaft and is offset from the driveshaft with the seal extending therebetween. The coupling is configured to magnetically couple the driveshaft to the impeller shaft.

### BRIEF DESCRIPTION OF THE DRAWINGS

FIG. 1 is a schematic illustration of a circulatory assist system including a circulatory assist device in accordance with embodiments of the disclosure;

FIG. 2A is a side view of a circulatory assist system in accordance with embodiments of the disclosure;

FIG. 2B illustrates the circulatory assist device of FIG. 8 with the wireless circulatory assist pump in a closed state (closed configuration);

FIG. 3 is a cross-sectional side view of an embodiment of the drive system 304 with a coupling for the circulatory assist device of FIGS. 2A and 2B, in accordance with embodiments of the disclosure;

FIG. 4 is a perspective view of an embodiment of components of the coupling of FIG. 3, in accordance with embodiments of the disclosure;

FIGS. 5A-5D are front views of embodiments of components of the coupling of FIG. 3;

FIG. 6 is a cross-sectional side view of an embodiment of the drive system the circulatory assist device of FIGS. 2A and 2B, in accordance with additional embodiments of the disclosure.

FIG. 7 is a perspective view of an embodiment of components of the coupling of FIG. 6, in accordance with additional embodiments of the disclosure;

FIGS. 8A-8D are front views of embodiments of components of the coupling of FIG. 6, in accordance with embodiments of the disclosure; and

FIG. 9 is a flowchart of a method of operating a circulatory assist device.

### DETAILED DESCRIPTION

Embodiments of the disclosure include circulatory assist systems devices and methods for operating circulatory assist systems and devices that can provide temporary and chronic circulatory support depending on the needs of the subject (e.g., a mammal, such as a human). Embodiments of circulatory assist device and components thereof in this disclosure may be utilized in combination with or integrated into other circulatory assist systems. For example, the circulatory assist device and components thereof of the disclosure may be utilized with or integrated into the circulatory assist pump and system described in U.S. 2021/0077687 A1 to Leonhardt and/or U.S. 2021/0008263 A1 to Leonhardt.

The circulatory assist device, and methods described herein involve a minimally invasive circulatory support platform that utilizes an aortic stent pump. For example, the circulatory support platform preferably uses a low profile, catheter-based technique. To implant the circulatory assist device within a subject for use, a medical professional (e.g., a surgeon) may incise the patient (e.g., in the groin region). The circulatory assist device and/or system may be in a closed (e.g., stowed) configuration and may remain in the closed configuration while the circulatory assist device and/or system is introduced into an artery, such as the femoral artery, of the subject. The circulatory assist device may be fed through a blood vessel in the subject to a desired location. For example, the circulatory assist device and/or system may be fed through an incision in the groin-area of the subject and into the subject’s femoral artery to a location just above the renal arteries within the subject’s aorta (e.g., the descending aorta above the subject’s kidneys). In certain embodiments, the desired location to position the circulatory assist device is in the ascending aorta. In response to being in the desired location, the circulatory assist device preferably transitions to an open (e.g., deployed) configuration and is activated to facilitate blood circulation through the subject’s blood vessel(s) (e.g., descending aorta) and/or organs (e.g., heart). For example, a stent cage may be deployed within and bracing the exterior walls of the subject’s aorta and the impeller may rotate within the stent cage to facilitate blood circulation.

Additionally, the circulatory assist devices described herein may include components, such as couplings, for transferring rotational motion to an impeller of a circulatory assist device. In various embodiments, the circulatory assist device includes one or more non-contact couplings (e.g., couplings in which components of the coupling are separated from one another) between independent shafts thereof. In various embodiments, the non-contact couplings (e.g., magnetic couplings) are configured to enable a first shaft to be separated from a second shaft and/or a material interposed between the first shaft and the second shaft, while enabling at least some rotational movement from the first shaft to transfer to the second shaft. As a non-limiting example, the first shaft is configured to be entirely contained within a closed (e.g., sealed) assembly and the second shaft is configured to be external to the closed assembly, and the non-contact coupling is configured to enable the second shaft to rotate in response to rotational movement of the first shaft. Accordingly, in various embodiments, the non-contact coupling(s) enable a shaft(s) within a closed assembly to rotationally couple to a shaft(s) outside of the closed assembly. Furthermore, the non-contact couplings may reduce friction, wear, and may correspondingly increase the longevity of the circulatory assist device. In addition, the non-contact couplings may eliminate the need for lubrication to mitigate friction and wear.

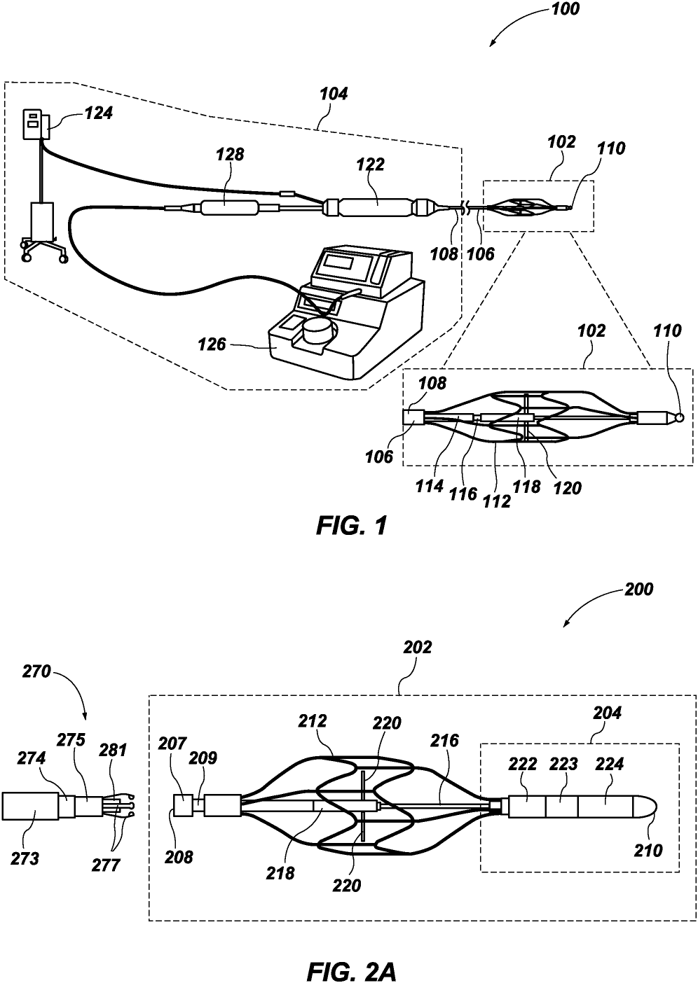

FIG. 1 is a schematic illustration of a circulatory assist system 100 including a circulatory assist device 102, according to embodiments of the disclosure. Referring to FIG. 1, in various embodiments, the circulatory assist system 100 includes a circulatory assist device 102 and a drive system 104. The drive system 104 may be substantially the same as the pump system described in U.S. 2021/0077687 A1 to Leonhardt and/or U.S. 2021/0008263 A1 to Leonhardt, the contents of each of which are incorporated herein by this reference.

The circulatory assist device 102 is configured to be inserted into a subject (e.g., a mammal such as a human) through in incision within the subject. The drive system 104 is configured to facilitate operation of the circulatory assist device 102. For example, in various embodiments, the circulatory assist device 102 is configured to expand after being inserted within the subject and/or to move (e.g., rotate) within the subject to facilitate circulation of fluids (e.g., blood) through blood vessels within the subject. Because the circulatory assist device 100 is configured to be inserted into the subject, in various embodiments, components and/or features of the circulatory assist device 102 are formed of and/or include biocompatible materials.

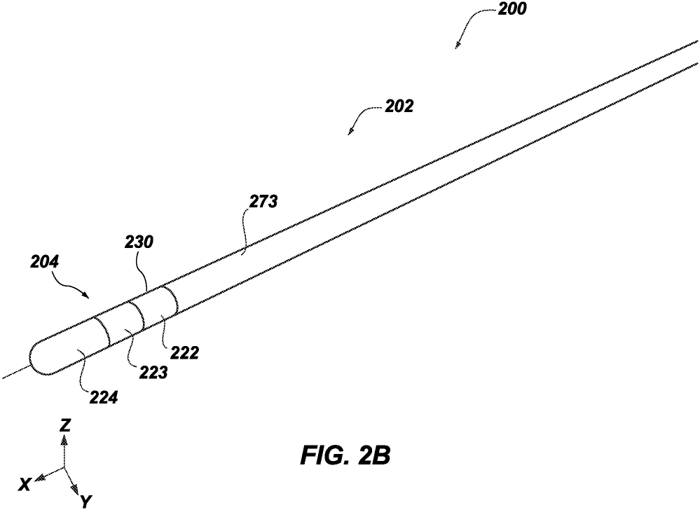

In various embodiments, the circulatory assist system 100 is a wired device in which drive system 104 remains external to the subject, while the circulatory assist device 102 is inserted into the subject. In at least some of these various embodiments, the drive system 104 is configured to facilitate insertion and/or placement of the circulatory assist device 102 within the subject. In various embodiments, the circulatory assist system 100 is a wireless system and is configured to be wirelessly driven. In some of these various embodiments, the drive system 104 is positioned within the circulatory assist device 102 as shown and described below with reference to FIGS. 2A and 2B.

Continuing with reference to FIG. 1, in various embodiments, the circulatory assist device 102 is configured to transition between an open (e.g., deployed state) and a closed (e.g., stowed or collapsed) state. In these various embodiments, the circulatory assist device 100 is introduced into the subject’s blood vessel(s) while in the closed state, and the circulatory assist device 100 is positioned in a desired location within the subject’s blood vessel(s), such as within the femoral artery of the subject. In these various embodiments, the circulatory assist device 102 is configured to transition to the open state once positioned in a desired location within the subject’s blood vessel(s). For example, In various embodiments, a medical professional (e.g., a doctor) changes a setting of the circulatory assist system 100 causing the circulatory assist device 102 to transition from the closed position to the open position and vice versa. Once the circulatory assist device 102 is expanded into the open position, the circulatory assist device 100 may be activated to begin assisting blood circulation through the subject’s blood vessel(s).

In various embodiments, the wired circulatory assist system 100 includes a driveline 108 configured to transfer the power provided by the drive system 104 to the circulatory assist device 102. In these various embodiments, the driveline 108 includes an exterior sheath 106 and a driveshaft 114. In various embodiments, the exterior sheath 106 is configured to cover the circulatory assist device 102 in the closed position and introduce the circulatory assist device 102 into the subject’s blood vessel while the circulatory assist device 102 is positioned therein.

In the wired circulatory assist system 100, the drive system 104 includes a motor 122, a power supply 124, a control unit 126, and a sheath controller 128. The motor 122 is configured to drive the circulatory assist device 102 in response to electrical energy applied thereto via the driveshaft 114. The power supply 124 (e.g., a medical grade UPS) facilitates transport and provides power to the various components of the circulatory assist system 100, such as the motor 122. The control unit 126 is configured to modify operation of the circulatory assist system 100, and in particular the circulatory assist device 102 via the motor 122 (e.g., adjust current) by enabling the user to enter desired operational parameters such as desired rotational speed of an impeller 118 (e.g., in revolutions per minute (RPM)) of the circulatory assist device 102. The sheath controller 128 is configured to control the extension/retraction of the exterior sheath 106 and/or one or more additional components of the circulatory assist system 100 to facilitate transitioning the circulatory assist device 102 between the open and closed states.

In various embodiments, the circulatory assist device 102 includes a stent cage 112 and an impeller 118. In various embodiments, in the closed state, the stent cage 112 is collapsed and radially interposed between the exterior sheath 106 and the driveshaft 114 and the impeller shaft 116. In the open state, the stent cage 112 is expanded radially outward relative to the closed state. In various embodiments, the stent cage 112 is formed of a material with a sufficient spring radial force within the material to maintain the shape thereof while expanded radially outward against an inner wall of the subject’s blood vessel, and the material of the stent cage 112 is sufficiently flexible to fold and/or compress when the exterior sheath 106 is moved axially over the stent cage 112.

The impeller 118 includes an impeller shaft 116 and one or more blades 120. Similar to the stent cage 112, the impeller 118 is configured to transition from a closed state, in which the impeller 118, including the one or more blades 120, is stowed within the outer sheath 106 to an open state, in which the one or more blades 120 extend outward from the impeller shaft 116/a body of the impeller 118. Furthermore, the impeller 118 while the stent cage 112 and the impeller 118 are in the open state, is configured to rotate about an axis of the impeller shaft 116, the impeller shaft 116 being operatively coupled to the driveshaft 114 and configured to rotate responsive to rotation of the driveshaft 114. The driveshaft 114 being driven by the motor 122 As will be described in greater detail below, in various embodiments, the coupling between the driveshaft 114 and the impeller shaft 116 is a non-contact coupling, such as a magnetic coupling, in which the driveshaft 114 and the impeller shaft 116 are rotationally coupled without direct physical contact. The rotational movement of the impeller 118 is configured to increase blood flow through the blood vessel(s) within the subject.

FIG. 2A is a side view of a circulatory assist system 200 in accordance with embodiments of the disclosure. In FIG. 2A and the associated description, functionally similar features (e.g., structures and materials) as those described above with reference to FIG. 1 are referred to with similar reference numerals incremented by 100. To avoid repetition, not all of the features shown in FIG. 2A are described in detail herein. Rather, unless described otherwise below, a feature in FIG. 2A designated by a reference numeral that is a 100 increment of the reference numeral of a previously described feature will be understood to be substantially similar to the previously described feature.

Referring now to FIG. 2A, in various embodiments, the circulatory assist system 100 includes a circulatory assist device 202 and a placement catheter 270. In these various embodiments, the circulatory assist device 202 is a wireless device configured to be positioned within and operate within a subject without a direct hardware connection to components exterior to the subject. In various embodiments and as will be described in further detail below, the placement catheter 270 is configured to insert and remove the circulatory assist device 202 into/from a blood vessel(s) of a subject.

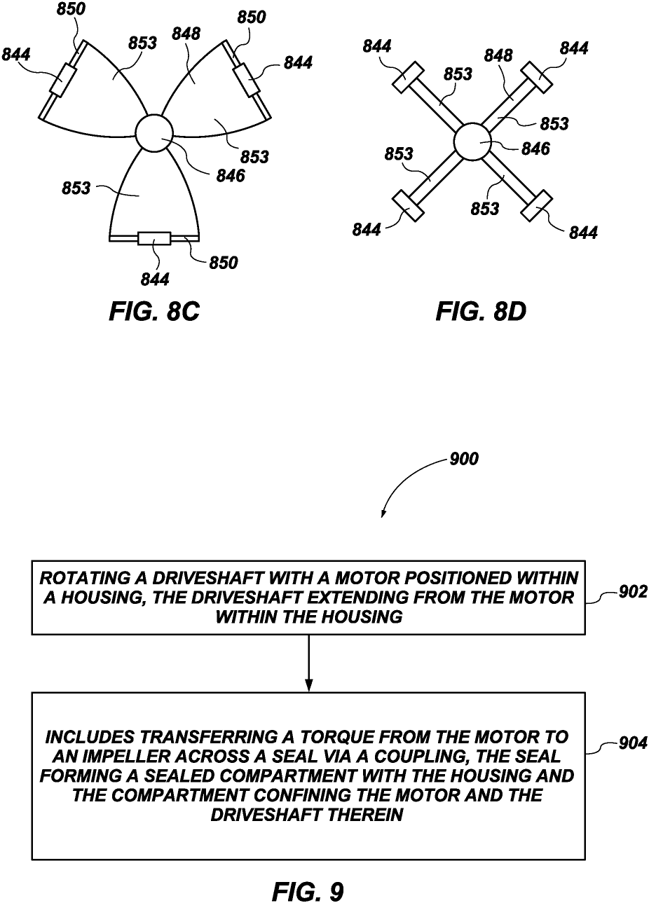

FIG. 2B illustrates the circulatory assist device of FIG. 8 with the wireless circulatory assist pump in a closed state (closed configuration). Referring to FIGS. 2A and 2B, placement catheter 270 is configured to connect to the circulatory assist device 202 to facilitate introduction and removal of the circulatory assist device 202 into a subject’s artery/blood vessel(s) (e.g., aorta). In some embodiments, the placement catheter 270 includes an outer sheath 273, an intermediate sheath 274, an inner sheath 275, and a gripper 277. The outer sheath 273, the intermediate sheath 274, and/or the inner sheath 275 may include an interior lining comprising expanded polytetrafluoroethylene (ePTFE).

The gripper 277 is positioned within the inner sheath 275 and is configured to extend out from the inner sheath 275 and grasp a gripping end 207 of the circulatory assist device 202 and maintain the grasp while components of the circulatory assist device 202, such as the stent cage 212 and the impeller 218 are positioned within the outer sheath 275 of the placement catheter 270. In some embodiments, the gripper 277 includes one or more fingers 279 surrounding an inner member 281 (e.g., a pin or ball). In some embodiments, the outer sheath 273 is configured to move axially relative to the intermediate sheath 274, and/or the inner sheath 275. Furthermore, in some embodiments, the gripper 277 is configured to move axially relative to each of the outer sheath 273, the intermediate sheath 274, and the inner sheath 275.

In some embodiments, the fingers 279 are biased radially outward. Thus, in response to being extended circumferentially relative to the inner member 275, the intermediate sheath 274, and the outer sheath 273, the tips of the fingers 279 extend radially outward and apart from one another. The fingers 279 are configured to receive the gripping end 207 of the circulatory assist device 202 therebetween. In some embodiments, the gripping end 207 includes a hole formed therein that is configured to receive the inner member 281 when the gripping end 207 is received within the fingers 279. In some embodiments, the hole in the gripping portion includes a taper that is configured to guide the coupling between the gripping end 207 and the gripper 277. Accordingly, the proximal end 208 of the circulatory assist device 202 is coupled to and aligned with the placement catheter 270. In various embodiments, at least one component chosen from the inner member 281 and the gripping end 207 (such as the portion of the gripping end forming the hole receiving the inner member 281) includes a magnetic material configured to form a magnetic connection between the inner member 281 and the gripping end 207.

In some embodiments, the outer sheath 273, intermediate sheath 274, and the inner member 275 are configured to move axially over the fingers 279, which biases the fingers 279 inward. Thus, in these embodiments, with the gripping end 207 received within the gripper 277, the fingers 281 are biased inward due to the relative movement between the gripper 277 and that of the inner member 275/intermediate sheath 274/outer sheath 273 which causes the fingers 281 to grip the gripping end 207, such as via an interference condition created by the radially inward biasing of the fingers 281, coupling the circulatory assist device 202 and the placement catheter 270 together.

In some embodiments, the outer sheath 273 includes an inner diameter that is larger than an outer diameter of the gripping end 207 and is configured to translate axially relative to the intermediate sheath 274, and the outer sheath 273 is configured to receive the gripping end 207, the impeller 218, and the stent cage 212 therein. The impeller 218 and the stent cage 212 are configured to collapse and stow within the outer sheath 273.

As the circulatory assist device 202 is wirelessly operated, in various embodiments, the circulatory assist device 202 includes an internal drive system 204 positioned within a casing 205 adjacent to the stent cage 212. In the embodiment illustrated, the drive system 204 is positioned at a distal end 210 of the circulatory assist device 202, opposite the proximal end 208/gripping end 207. The proximal end 208 of the circulatory assist device 200 may be oriented closer to the incision in which the circulatory assist device 200 is inserted into the subject than the distal end 210.

In various embodiments, the drive system 204 of the circulatory assist device 200 includes a motor 222, circuitry 223, and a power supply 224 positioned within a housing 230. In various embodiments, the circuitry 223 is adjacent to the motor 222 and the power supply 224 is adjacent to the circuitry 223. The motor 222 is configured to rotate a drive shaft coupled to the impeller shaft 216 of the impeller 218 to facilitate blood circulation within the subject’s blood vessel(s). For example, in various embodiments, the motor 222 is configured to rotate up to about 32,000 RPM (e.g., under no load). Additionally, in various embodiments, under normal operating conditions, the motor 222 is configured to rotate at a speed within a range of from about 4000 RPM to about 15,000 RPM, such as from about 6,000 RPM to about 12,000 RPM (e.g., about 9000 RPM).

In certain embodiments, the motor initially operates at an initial lower speed (e.g., from as low as 1,500 RPM to 4,000 RPM), but after stabilization of the patient is increased to about 6,000 RPM to about 12,000 RPM (e.g., about 10,000 RPM).

In various embodiments, the motor 222 is a miniature brushless direct current (DC) motor. For example, in some of these various embodiments, the motor 222 is a miniature brushless DC motor such as available under the tradename “EC6” from Maxon Precision Motors, Inc. of Foster City, California USA.

In various embodiments, the circuitry 223 includes a wireless charging circuit, a communications circuit, and a control circuit. The wireless charging circuit produces an electric current in response to an applied electric field, magnetic field, and/or electromagnetic field, which, in various embodiments is utilized to charge the power supply 224. For example, in various embodiments, the wireless charging circuit includes an induction coil configured to receive energy via inductive coupling. The wireless charging circuit is electrically coupled to the power supply 224 and is configured to provide the energy received via inducting coupling to the power supply 224 for use by the motor 222. In various embodiments, the wireless charging circuit includes one or more antennas configured to receive energy via electromagnetic waves (e.g., radio waves) received thereby.

In various embodiments, the communication circuit is configured to send and receive data via wireless communication. For example, in some of these various embodiments, the communication circuit is configured to send and receive data utilizing radio communication (e.g., Wi-Fi, BluetoothTM, etc.). In various embodiments, the communication circuit is configured to communicate over the Medical Implant Communication System (MICS) band, a low-power, short-range, high-data-rate band from 401 MHz to 406 MHz. In some of these embodiments, the communication circuit is utilized to send data collected from one or more sensors of the wireless circulatory assist device 202. For example, in some embodiments, the communication circuit is utilized to send data relating to the rotational speed of the pump, upstream and downstream fluid pressures, battery charge status, motor status, impeller status, and/or other measured conditions.

The control circuit is configured to control certain operations of the wireless circulatory assist device 200. In some embodiments, the control circuit may be utilized to control the rotational speed of the motor 222, the deployment of the impeller blades 220, the stowing of the impeller blades 220, and/or other operations of the circulatory assist device 200.

In various embodiments, the circulatory assist device 202 includes one or more application-specific integrated circuit (“ASIC”) chips. For example, in some of these various embodiments, one or more of the charging circuit, the communication circuit, and the control circuit are provided as one or more ASIC chips.

In various embodiments, the power supply 224 includes one or more batteries. In various embodiments, the power supply 224 includes a rechargeable battery, such as a lithium-ion battery. For example, in various embodiments, the power supply 224 includes a 3 milliamp hour (mAh) lithium-ion battery available under the tradename “CONTIGO” from Eagle Picher Technologies of Joplin, Missouri USA. For another example, in various embodiments, the power supply 224 includes a 3 mAh lithium-ion battery available under the tradename “MICRO3-QL0003B” from Quallion LLC of Sylmar, California USA. It will be understood, however, that the power supply 224 may be of any suitable chemistry and/or type, including non-chemical electric power storage devices, such as a capacitor (e.g., a supercapacitor, ultracapacitor, or double-layer capacitor).

Although illustrated as at or near the distal end 210 of the circulatory assist device, the motor 222, the circuitry 223, and the power supply 224 may all be located in other locations of the circulatory assist device 202, such as at or near the proximal end 208. Additionally, because the circulatory assist device 202 may remain within the subject for extended periods of time, in various embodiments, the motor 222, the circuitry 223, and/or the power supply 224 are positioned within the housing 230 to prevent contact with the subject’s bodily fluids (e.g., blood). For example, in various embodiments, the housing 230 is hermetically sealed to prevent blood from infiltrating into the motor 222, the circuitry 223, and/or the power supply 224.

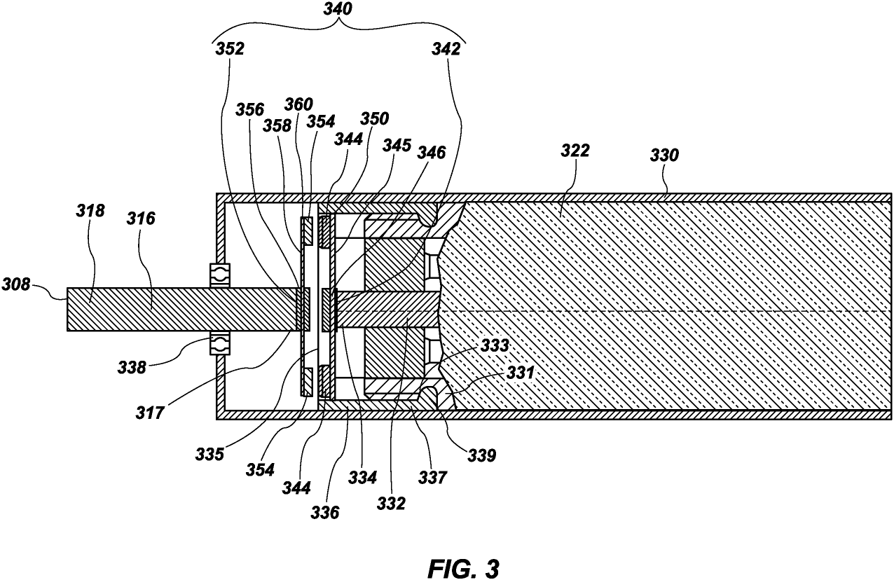

FIG. 3 is a cross-sectional side view of an embodiment of the drive system 304 with a coupling 340 for the circulatory assist device of FIGS. 2A and 2B, in accordance with embodiments of the disclosure. In FIG. 3 and the associated description, functionally similar features (e.g., structures, materials) as those described above with reference to FIG. 2 are referred to with similar reference numerals incremented by 100. To avoid repetition, not all features shown in FIG. 3 are described in detail herein. Rather, unless described otherwise below, a feature in FIG. 3 designated by a reference numeral that is a 100 increment of the reference numeral of a previously described feature will be understood to be substantially similar to the previously described feature.

Referring now to FIG. 3, in various embodiments, the drive system 304 includes a driveshaft 332 extending from the motor 322 and configured to be rotationally driven by the motor 322 The driveshaft 332 includes a shaft end 334 distal from the motor 322 and oriented toward an end 317 of the impeller shaft 316. In various embodiments, the end 317 of the impeller shaft 316 extends partially into the housing 330 with a gap between the end 317 of the impeller shaft 316 and the shaft end 334 of the driveshaft 332, such that the impeller shaft 316 and the driveshaft 332 are mechanically de-coupled (i.e., physically do not touch or joined together by mechanical mechanisms). In various embodiments, the housing 330 includes an opening formed therein for the impeller shaft 316 to pass therethrough with radial bearings 338 positioned therein between the impeller shaft 316 and a body of the housing 330.

In various embodiments, the housing 330 includes a seal 336 configured to seal one or more compartments within the housing 330 from the environment. In some of these various embodiments, the seal 336 is positioned between the driveshaft 332 and the impeller shaft 316. For example, in various embodiments, the seal 336 includes a cylindrical cap covering one end of the motor 322 and forms a barrier that separates the motor 322 and the driveshaft 332 from the impeller shaft 316. In various embodiments, the seal 336 forms a hermetic seal with a body of the housing 330 to hermetically seal one or more compartments of the housing 330, the one or more compartments including components, such as the motor 322, therein.

In various embodiments, the seal 336 includes a cap portion 335 with a disk shape and a seal portion 337 that includes a hollow cylinder shape that extends towards the motor 322 and that contacts an inner surface of the body of the housing and forms a seal therewith. In various embodiments, the seal portion includes a lip 339 extending radially inward from the hollow cylinder shape. In various embodiments, the housing includes a seal mount 331 positioned within the body and connected thereto. The seal mount 331 includes a groove 333 formed therein. In an assembled state, the lip 339 is received and mated to the groove 333 to hold the seal 336 within the body of the housing 330. In some of these embodiments, the seal 336 includes a biocompatible material.

The motor 322 is configured to receive electrical energy from the power supply (e.g., the power supply 124, 224) to rotate the driveshaft 332. The drive system 304 includes a coupling 340 that magnetically couples the drive shaft 332 to the impeller shaft 316 to transfer the torque from the motor 322 to the impeller to cause the impeller to rotate. The coupling 340 is a non-contact coupling (i.e., mechanically de-coupled with no mechanical contact between the driveshaft 332 and the impeller shaft 316) configured to transfer at least some (e.g., some, or all) rotational motion from the driveshaft 332 to the impeller shaft 316. In various embodiments, the rotational motion/torque of the driveshaft 332 is transferred to the impeller shaft 316 utilizing magnetism. In various embodiments, the driveshaft 332 and the impeller shaft 316 are aligned axially and the coupling 340 is configured to create magnetic field and/or magnetic force between impeller shaft 316 and the driveshaft 332. In various embodiments, the coupling 340 co-axially couples the impeller shaft 316 and the driveshaft 332 allowing torque applied to the driveshaft 332 to be transferred to the impeller shaft 316.

In various embodiments, the coupling 340 (e.g., non-contact coupling) includes a first coupler 342 and a second coupler 352. The first coupler 342 is configured to be secured to a first shaft (e.g., one of the driveshaft 332 and the impeller shaft 316), and the second coupler 352 is configured to be secured to a second shaft (e.g., the other of the driveshaft 332 and the impeller shaft 316). In various embodiments, the first coupler 342 includes a body 348 and one or more magnets 344 (e.g., a first set/array of the magnets 344) secured to the body 348, and the second component 352 includes a body 358 and one or more magnets 354 (e.g., a second set/array of magnets 354) secured to the body 358. In various embodiments, the first coupler 342 includes an inner portion 346 of the body 348 joined to the first shaft joining the first coupler 342 to the first shaft. The second coupler 352 includes an inner portion 358 of the body 356 secured to the second shaft joining the second coupler 352 to the second shaft.

In various embodiments, the first coupler 342 and the second coupler 352 include similar sizes, shapes, and arrangements to align the magnets 344 and 354 for the magnetic coupling thereof.

In some embodiments, the magnets 344 are secured proximate an exterior edge 350 of the body 348, distal to the inner portion 346. In various embodiments, the exterior edge 350 and/or a portion of the magnets 344 defines a rotational diameter of the first coupler 342. In various embodiments, the magnets 344 are secured to the body 348 at a position opposite and facing away from the motor 322, and facing towards the seal 336. The magnets 344 may include any magnetic material. In some embodiments, the magnets 344 include neodymium (Nd) and/or samarium-cobalt (SmCo). In some embodiments, the magnets 354 are secured proximate an exterior edge 360 of the body 358, distal to the inner portion 356. In various embodiments, the exterior edge 360 and/or a portion of the magnets 354 defines a rotational diameter of the second coupler 352. In various embodiments, the magnets 354 are secured to the body 358 at a position opposite and facing away from the blades of the impeller, and facing towards the seal 336. The magnets 354 may include any magnetic material. In some embodiments, the magnets 354 include neodymium (Nd) and/or samarium-cobalt (SmCo). In one or more embodiments, the magnets 354 include the same material as the magnets 344. In additional embodiments, the magnets 354 comprise a different material than the magnets 344.

In various embodiments, the magnets 344 of the first coupler 342 and the magnets 354 of the second coupler 352 form magnetic pairs, each pair including one of the magnets 344 of the first coupler 342 and one of the magnets 354 of the second coupler 352. The magnetic field and magnetic force between corresponding pairs of the magnets 344 and the magnets 354 combined are substantially strong enough such that rotation of the driveshaft 332 and the magnets 344 of the first coupler 342 secured to the driveshaft 332 causes the magnets 354 of the second coupler 352 secured to the impeller shaft 316 to rotate (i.e. the magnetic force between the magnets 344 and the magnets 354 applies a torque to the second coupler 352 causing the second coupler 352 and the impeller shaft 316 to rotate). As illustrated in FIG. 3, in various embodiments, the magnets 344 of the first coupler 342 are axially offset and radially aligned with the magnets 354 of the second coupler 352. Accordingly, a magnetic force between the magnets 344 and the magnets 354 is applied in the axial direction (and the circumferential direction upon rotation of the magnets 344), which may apply axial forces on the impeller shaft 316 and the driveshaft 332.

In various embodiments, the motor 322 includes a thrust bearing to overcome the axial force resulting from the magnets 344 and the magnets 354, and inhibit or prevent axial movement of the driveshaft 332. The thrust bearing is configured to control the axial position of the driveshaft 332 to limit axial movement thereof, and in particular an axial gap between the seal 336 and the driveshaft 332, the first coupler 342, and/or the magnets 344. The axial gap between the seal 336 and the magnets 344 may prevent contact therebetween, which may reduce wear and may improve the longevity of the coupling 340.

As noted above, in various embodiments, the drive system 304 includes radial bearings 338 positioned between (e.g., radially between) the impeller shaft 316 and the housing 330. In various embodiments, the radial bearings 338 are connected to the housing 330 and configured to radially support one end of the impeller shaft 316 to reduce and/or prevent radial movement of the impeller shaft 316. In various embodiments, the drive system 304 also includes a thrust bearing to control the axial position of the impeller shaft 316. The thrust bearing is configured to oppose the axial force applied to the impeller shaft 316 by the magnetic forces between the magnets 344 and the magnets 354 and may limit or prevent the impeller shaft 316, the magnets 354, and/or the second coupler 352 from contacting the seal 336. The axial gap between the magnets 354 and the seal 336 may prevent contact therebetween, which may reduce wear and may improve the longevity of the coupling 340.

As described previously herein, the coupling 340 is a magnetic coupling that does not include mechanical contact between the impeller shaft 316 and the driveshaft 332, which may eliminate the need for lubrication, may reduce wear of the circulatory assist device 300, and may allow the housing 330 to be hermetically sealed with the seal 336. Such a seal may prevent contact between the motor 322 and the subject’s bodily fluids.

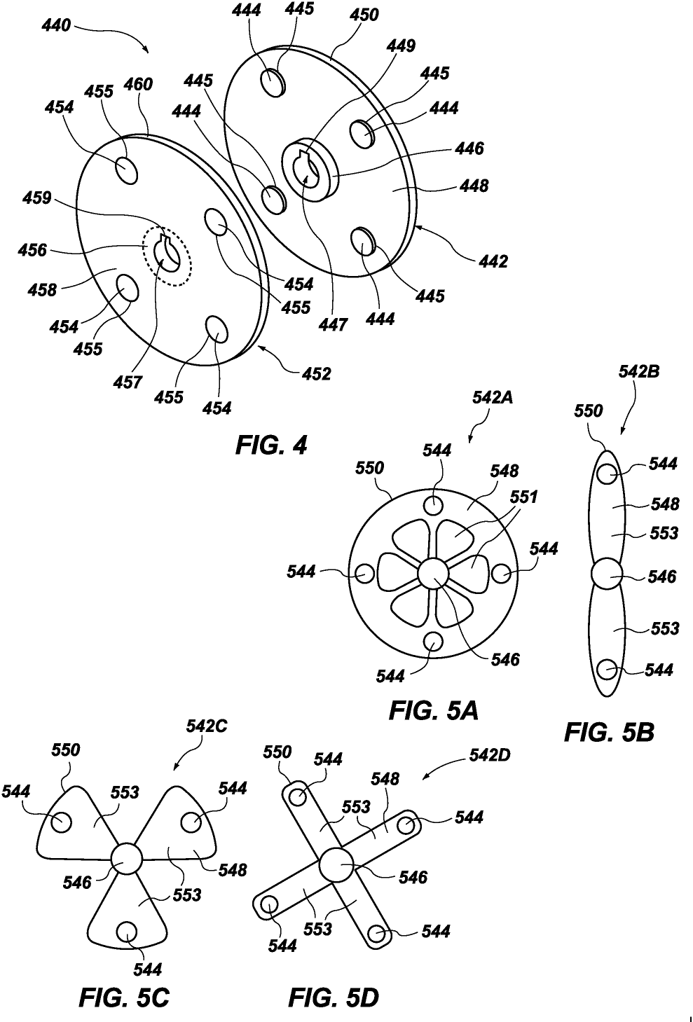

FIG. 4 is a perspective view of an embodiment of components of the coupling 440 of FIG. 3, in accordance with embodiments of the disclosure. In FIG. 4 and the associated description, functionally similar features (e.g., structures, materials) as those described above with reference to FIG. 3 are referred to with similar reference numerals incremented by 100. To avoid repetition, not all features shown in FIG. 4 are described in detail herein. Rather, unless described otherwise below, a feature in FIG. 4 designated by a reference numeral that is a 100 increment of the reference numeral of a previously described feature will be understood to be substantially similar to the previously described feature.

Referring now to FIG. 4, in various embodiments, each of the first coupler 442 and the second coupler 452 of the coupling 440 includes a substantially cylindrical shape. For example, in some of these various embodiments, each of the first coupler 442 and the second coupler 452includes a cylindrical disc shape (e.g., a solid right circular cylinder with a thickness significantly smaller than the diameter thereof, without limitation). In some of these various embodiments, the first coupler 442 includes the same shape as the second coupler 452. In various other embodiments, the first coupler 442 includes a shape that is different than the second coupler 452.

As described above, in various embodiments, the first coupler 442 includes one or more magnets 444 secured to the body 448, such as proximate the exterior edge 450 of the body 448. In some embodiments, the exterior edge 450 defines an outer diameter of the cylindrical shape of the first coupler 442. In various embodiments, the first coupler 442 includes a mounting region 445 for each of the magnets 444 formed in the body 448 and configured to receive a respective magnet 444 therein for securing the respective magnet 444 to the body 448. In various embodiments, each of the mounting regions 445 includes a hole chosen from a through-hole and a blind-hole with a size and shape corresponding to that of the corresponding magnet 444. In various embodiments, the magnets 444 protrude from a surface of the body 448.

As noted above, the inner portion 446 is configured to secure the first coupler 442 to the first shaft (e.g., one of the driveshaft 332 and impeller shaft 316). In various embodiments, the inner portion 446 includes an opening 447 formed therein that is configured to receive the first shaft. In various embodiments, the inner portion 446 includes a slot 449 formed therein extending radially from the opening defining a key for the first shaft. In these embodiments, the first shaft includes a radial protrusion that is received in the slot 449. In various embodiments, the inner portion 446 protrudes axially from the body 448 at least in one axial direction. In the embodiment illustrated, the inner portion 446 protrudes towards the second coupler 452 when installed on the first shaft.

The first coupler 442 is secured to the first shaft to inhibit and/or prevent relative rotational movement therebetween. In various embodiments, the inner portion 446 is joined to the first shaft using biocompatible material adhesives (e.g., glue, epoxy), using biocompatible material fasteners (e.g., bolts, screws, pins, nails), and/or by fusing (e.g., welding, brazing) the first coupler 442 to the first shaft.

As described previously herein, in various embodiments, the second coupler 452 includes one or more magnets 454 secured to the body 458, such as proximate the exterior edge 460 of the body 458. In some embodiments, the exterior edge 460 defines an outer diameter of the cylindrical shape of the second coupler 452. In various embodiments, the second coupler 452 includes a mounting region 455 for each of the magnets 454 formed in the body 458. In various embodiments, each of the mounting regions 455 includes a hole chosen from a through-hole and a blind-hole with a size and shape corresponding to that of the corresponding magnets 454. In various embodiments, the magnets 454 protrude from a surface of the body 448.

As noted previously herein, the inner portion 456 is configured to secure the second coupler 452 to the second shaft (e.g., the other of the driveshaft 332 and impeller shaft 316). In various embodiments, the inner portion 456 includes an opening 457 formed therein that is configured to receive the second shaft. In various embodiments, the inner portion 456 includes a slot 459 formed therein extending radially from the opening defining a key for the second shaft. In these embodiments, the second shaft includes a radial protrusion that is received in the slot 459. In various embodiments, the inner portion 456 protrudes axially from the body 458 at least in one axial direction, such as towards the first coupler 442 when installed on the second shaft.

The second coupler 452 is secured to the second shaft to inhibit and/or prevent relative rotational movement therebetween. In various embodiments, the inner portion 456 is joined to the second shaft using biocompatible material adhesives (e.g., glue, epoxy), using biocompatible material fasteners (e.g., bolts, screws, pins, nails), and/or by fusing (e.g., welding, brazing) the second coupler 452 to the second shaft.

The magnets 444 and 454 are sized, shaped, and positioned to ensure a magnetic coupling between the first coupler 442 and the second coupler 452, such that the first coupler 442 and the second coupler 452 rotate together. In various embodiments, the magnets 444 are positioned with rotational symmetry on the body 448, and the magnets 454 are positioned with rotational symmetry on the body 458. In some embodiments, the magnets 444 of the first coupler 442 are substantially the same size as the magnets 454 of the second coupler 452. In additional embodiments, the magnets 444 of the first coupler 442 are a different size than the magnets 454 of the second coupler 452. Additionally, one or more of the magnets 444 of the first coupler 442 may be different shapes and/or sizes from one another. Similarly, one or more magnets 454 of the second coupler 452 may be different shapes and/or sizes from one another.

While the embodiment illustrated in FIG. 4 includes the body 448 and the body 458 with a cylindrical shape, as will be discussed in further detail below, in various embodiments, the body 448 and the body 458 includes other shapes, such as those illustrated in FIGS. 5A-5D. In some embodiments, the first coupler 442 includes a number of magnets 444 that is the same as a number of magnets 454 of the second coupler 452.

FIGS. 5A-5D are front views of embodiments of components of the coupling of FIG. 3. In FIGS. 5A-5D and the associated description, functionally similar features (e.g., structures, materials) as those described above with reference to FIG. 4 are referred to with similar reference numerals incremented by 100. To avoid repetition, not all features shown in FIGS. 5A-5D are described in detail herein. Rather, unless described otherwise below, a feature in FIG. 4 designated by a reference numeral that is a 100 increment of the reference numeral of a previously described feature will be understood to be substantially similar to the previously described feature. The following description in reference to the couplers 542A-542D, applies to both a first coupler (e.g., the first coupler 342, 442) and also applies to the second coupler (e.g., the second coupler 352, 452) of couplings described herein (e.g., the coupling 340, 440).

Referring collectively to FIGS. 5A-5D, the couplers 542A-542D viewed in an axial direction, include a variety of shapes (e.g., circular, rectangular, triangular). Additionally, the couplers 542A-542D include a body 548 and an inner portion 546 configured to secure the respective coupler 542A-542D to a shaft (driveshaft or impeller shaft).

Referring now to FIG. 5A, in various embodiments, the coupler 542A includes a wheel and spoke shape. The body 548 includes an exterior ring and spokes extending between the exterior ring and the inner portion 546. The spokes define openings 551 between the spokes. In the embodiment illustrated, the coupler 542A includes four magnets 544 proximate the exterior edge 550. However, other numbers of magnets 544 and shapes of magnets 544 are also contemplated, such as a singular annular magnet, multiple annular sectors, and the like.

Referring now to FIGS. 5B-5D, in various embodiments, the body 548 defines blades 553 extending from the inner portion 546 to the exterior edge 550. In these embodiments, one or more magnets 544 is secured to each blade 553. In some of these embodiments, the magnets 544 are secured proximate the exterior edge 550 of a respective blade 553. The exterior edge 550 of the blades 553 defines a rotational diameter of the couplers 542A-542D.

Referring to FIG. 5B, in various embodiments, the coupler 542B includes two blades 553 extending about at 180 degrees from one another. In various embodiments, each blade 553 includes an elliptical shape. In various embodiments, the coupler 542B includes two magnets 544, one magnet 544 secured to each blade 553.

Referring to FIG. 5C, in various embodiments, the coupler 542C includes three blades 553 extending about 120 degrees apart from one another. In various embodiments, each blade 553 includes a triangular/circular sector shape. In various embodiments, the coupler 542C includes three magnets 544, with one magnet 544 secured to each blade 553.

Referring to FIG. 5D, in various embodiments, the coupler 542D includes four blades 553 extending about 90 degrees apart from one another. In various embodiments, each blade 553 includes a rectangular shape. In various embodiments, the coupler 542D includes a four magnets 544, with one magnet 544 secured to each blade 553.

Although illustrated as including a magnet on each blade 553, the component 542B-542D may include more blades 553 than magnets 544, or more magnets 544 than blades 553. For example, one or more of the blades 553 may include multiple magnets 544. Additionally, one or more of the blades 553 may include no magnets 544. Further, while each of the couplers 542A-542D is illustrated with blades of particular shapes, in various embodiments, the couplers include different combinations of blade numbers and blade shapes.

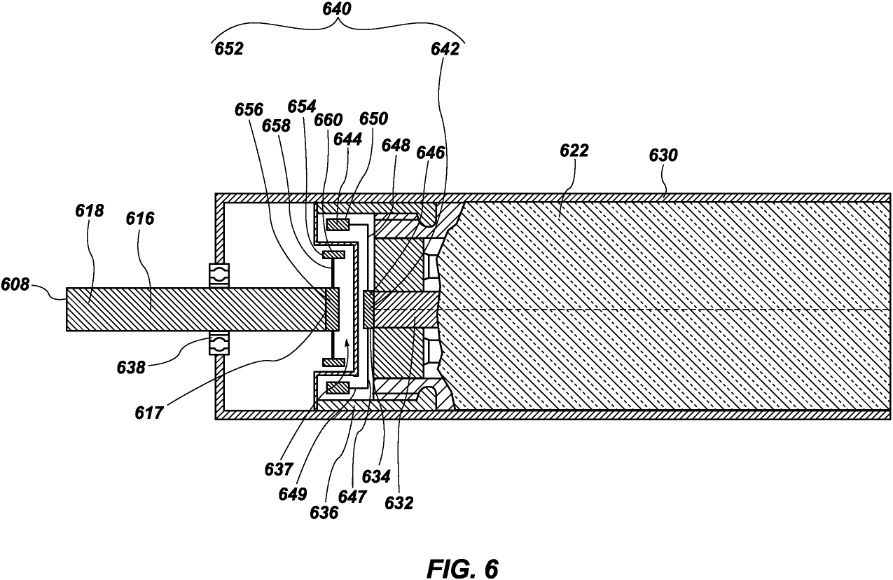

FIG. 6 is a cross-sectional side view of an embodiment of the drive system for the circulatory assist device of FIGS. 2A and 2B, in accordance with embodiments of the disclosure. In FIG. 6 and the associated description, functionally similar features (e.g., structures, materials) as those described above with reference to FIG. 3 are referred to with similar reference numerals incremented by 300. Unless described otherwise below, a feature in FIG. 6 designated by a reference numeral that is a 300 increment of the reference numeral of a previously described feature will be understood to be substantially similar to the previously described feature.

Referring now to FIG. 6, similar to the embodiments of FIG. 3, the drive system 604 includes a driveshaft 632 extending from the motor 622 configured to be rotationally driven by the motor 622. The driveshaft 632 includes a shaft end 634 distal from the motor 622 and oriented toward an end 617 of the impeller shaft 616. In various embodiments, the end 617 of the impeller shaft 616 extends partially into the housing 630 with a gap between the end 617 of the impeller shaft 616 and the shaft end 634 of the driveshaft 632, such that the impeller shaft 616 and the driveshaft 632 are mechanically de-coupled (i.e. physically do not touch or joined together by mechanical mechanisms). In various embodiments, the housing 630 includes an opening formed therein for the impeller shaft 616 to pass therethrough with radial bearings 638 positioned therein between the impeller shaft 616 and a body of the housing 630.

In various embodiments, the housing includes a seal 636 configured to seal one or more compartments within the housing 330 from the environment. In some of these various embodiments, the seal 336 is positioned between the driveshaft 632 and the impeller shaft 616. In various embodiments, the seal 636 includes a cap portion 635 configured to cover one end of the motor 622. In various embodiments, the cap portion forms a cavity 637 centrally located with an outer portion of the cap portion 635 forming an annular ring protruding beyond the cavity 637, the cavity 637 being open to the exterior of the housing 630, while the annular ring is open to the interior of the housing 630. In various embodiments, the seal 636 forms a hermetic seal around the motor 622 with interior surfaces of the housing 630. In some embodiments, the seal 636 includes a biocompatible material.

The motor 622 is configured to receive electrical energy from the power supply (e.g., the power supply 124, 224) to rotate the driveshaft 632. The drive system 604 includes a coupling 640 that magnetically couples the driveshaft 632 to the impeller shaft 616 to transfer the torque from the motor 622 to the impeller to cause the impeller to rotate. The coupling 640 is a non-contact coupling (i.e., mechanically de-coupled with no mechanical contact between the driveshaft 632 and the impeller shaft 616) configured to transfer at least some (e.g., some, or all) rotational motion from the driveshaft 632 to the impeller shaft 616. In various embodiments, the rotational motion/torque of the driveshaft 632 is transferred to the impeller shaft 616 utilizing magnetism. In various embodiments, the driveshaft 632 and the impeller shaft 616 are aligned axially and the coupling 640 is configured to create magnetic field and/or magnetic force between the impeller shaft 616 and the driveshaft 632. In various embodiments, the coupling 640 co-axially couples the impeller shaft 616 and the driveshaft 632 allowing torque applied to the driveshaft 632 to be transferred to the impeller shaft 616.

In various embodiments, the coupling 640 (e.g., non-contact coupling) includes a first coupler 642 and a second coupler 652. The first coupler 642 is configured to be secured to a first shaft (e.g., one of the driveshaft 632 and the impeller shaft 616), and the second coupler 652 is configured to be secured to a second shaft (e.g., the other of the driveshaft 632 and the impeller shaft 616).

In various embodiments, the first coupler 642 includes one or more magnets 644 (e.g., a first set of magnets 644) and the second coupler 652 includes one or more magnets 654 (e.g., a second set of magnets 654). As will be described in further detail below, in various embodiments, the first coupler 642 and the second coupler 652 are configured to axially align the magnets 644 and magnets 654 with a radial gap therebetween. In various embodiments, the magnets 644 and magnets 654 are also circumferentially aligned.

Continuing with reference to FIG. 6, in various embodiments, the first coupler 642 a body 648 and an inner portion 646. The body 648 is configured to hold the magnets 644 in a position radially outward and axially aligned with the magnets 654. In various embodiments, the body 648 includes a disk portion 647 extending radially outward from the inner portion 646 and an extension portion 649 extending axially from the disk portion 647 in a direction away from the motor 622. In various embodiments, the extension portion 649 includes a hollow cylinder shape. In some of these embodiments, the hollow cylinder shape extends from an outer edge of the disk portion. In various embodiments, the magnets 644 are secured to the extension portion 649 axially offset from the inner portion 646 and the disk portion 647. In some of these various embodiments, the magnets 644 are joined to the extension portion at an exterior edge 650 thereof. In other various embodiments, the magnets 644 are joined to an inner radial surface of the extension portion 649, such as proximate to the exterior edge 650. In various embodiments, the extension portion 649 extends into the annular ring of the cap portion 635 with the magnets 644 positioned therein. The magnets 644 may include any magnetic material. In some embodiments, the magnets 644 include neodymium (Nd) and/or samarium-cobalt (SmCo).

In various embodiments, the second coupler 652 is positioned within the cavity 637 of the cap portion 635 and axially aligned with the magnets 6446. The second coupler 652includes a body 658 and an inner portion 656In various embodiments, the magnets 654 are joined to the body 658, such as at or proximate the exterior edge 660 of the body 658. In various embodiments, the magnets 654 are axially and circumferentially aligned with the magnets 644. The magnets 654 may include any magnetic material. In some embodiments, the magnets 654 include neodymium (Nd) and/or samarium-cobalt (SmCo). In one or more embodiments, the magnets 654 include the same material as the magnets 644. In additional embodiments, the magnets 654 include a different material than the magnets 644.

In various embodiments, the magnets 644 of the first coupler 642 and the magnets 654 of the second coupler 652 form magnetic pairs, each pair including one of the magnets 644 of the first coupler 642 and one of the magnets 654 of the second coupler 652. The magnetic field and magnetic force between corresponding pairs of the magnets 644 and the magnets 654 combined are substantially strong enough such that rotation of the first shaft and the magnets 644 of the first coupler 342 causes the magnets 654 of the second coupler 652 to rotate (i.e. the magnetic force between the magnets 644 and the magnets 654 applies a torque to the second coupler 652 causing the second coupler 652 and the second shaft to rotate) As illustrated in FIG. 6, in various embodiments, the magnets 644 and the magnets 654 are axially aligned and radially offset.

In various embodiments, the drive system 604 includes bearings, such as radial bearings 638 and thrust bearings to maintain stability of the driveshaft 632 and the impeller shaft 616, which may be the same or similar to the bearings described above with regards to FIG. 3.

As described above, the coupling 640 is a magnetic coupling that does not include mechanical contact between the impeller shaft 616 and the driveshaft 632, which may eliminate the need for lubrication, may reduce wear of the circulatory assist device 600, and may allow the housing 630 to be hermetically sealed with the seal 636. Such a seal may prevent contact between the motor 622 and the subject’s bodily fluids.

While the embodiments described with regards to FIG. 6 include magnets 644 that are axially aligned and radially offset from magnets 654 and the embodiments described with regards to FIG. 3 include magnets 344 that are axially aligned and radially offset from magnets 354, in various embodiments, the coupling 340, 640 includes some magnets 344 that are axially aligned and radially offset from magnets 354 and some magnets 644 that are axially aligned and radially offset from magnets 654. Thus, in various embodiments, first magnets are in a position relative to second magnets chosen from (1) axially offset and radially aligned, (2) axially aligned and radially offset, and (3) at least one first magnet axially offset and radially aligned relative to at least one second magnet and at least another first magnet axially aligned and radially offset from at least another second magnet.

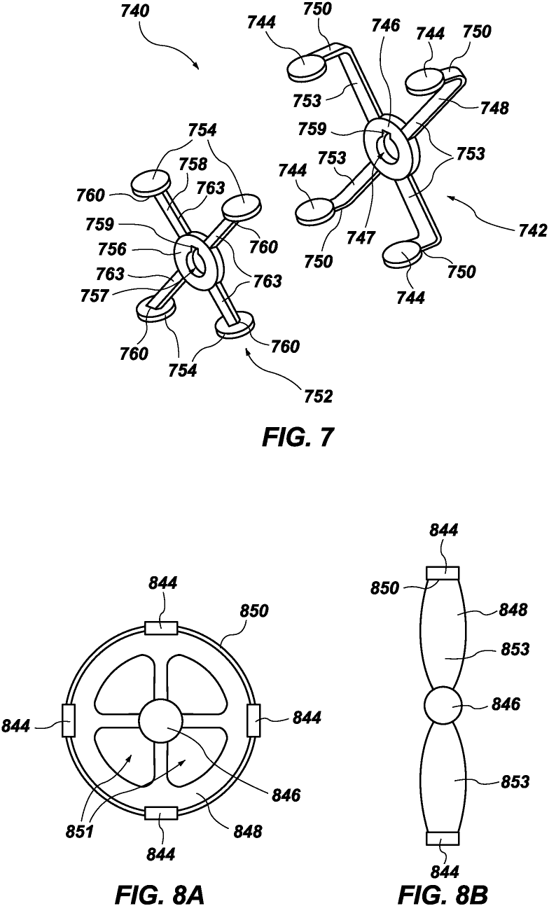

FIG. 7 is a perspective view of an embodiment of components of the coupling of FIG. 6, in accordance with additional embodiments of the disclosure. Referring now to FIG. 7, in various embodiments, each of the first coupler 742 and the second coupler 752 of the coupling 740 includes a body 748, 758 including multiple blades 753, 763 extending radially outward from the inner portion 746,756. In the embodiment illustrated in FIG. 7, the body 748, 758 includes four blades 753, 763 in the shape of an “X,” with each of the blades separated by about 90 degrees. In various embodiments, each body 748 includes an arm 749 extending axially from an end of the respective blade 753. In various embodiments, the first coupler 742 includes a number of blades 753 that is the same as a number of blades 763 of the second coupler 752. In other embodiments, the first component 742 includes a number of blades 753 that is different than a number of blades 763 of the second coupler 752.

In various embodiments, the magnets 744 are joined to the body 748 at the arms 649, such as at an exterior edge 750 thereof or on a radially inner surface thereof.

As noted above, the inner portion 746 is configured to secure the first coupler 742 to the first shaft (e.g., one of the driveshaft 632 and impeller shaft 616). In various embodiments, the inner portion 746 includes an opening 747 formed therein that is configured to receive the first shaft. In various embodiments, the inner portion 746 includes a slot 749 formed therein extending radially from the opening defining a key for the first shaft. In these embodiments, the first shaft includes a radial protrusion that is received in the slot 749. In various embodiments, the inner portion 746 protrudes axially from the body 748 at least in one axial direction. In the embodiment illustrated, the inner portion 746 protrudes towards the second coupler 752 when installed on the first shaft.

In various embodiments, the first coupler 742 is secured to the first shaft using biocompatible material adhesives (e.g., glue, epoxy), using biocompatible material fasteners (e.g., bolts, screws, pins, nails), and/or by fusing (e.g., welding, brazing) the first coupler 742 to the first shaft.

As described previously herein, in various embodiments, the second coupler 752 includes one or more magnets 754 secured to the body 758, such as proximate the exterior edge 760 of the body 758. In some embodiments, the exterior edge 760 defines an outer diameter of the second coupler 752. In various embodiments, the magnets 754 are secured to ends of the blades 753 distal to the inner portion 756 and positioned radially inward from the magnets 744 of the first coupler 742. In various embodiments, the magnets 754 extend outward (e.g., radially and/or axially outward) from the exterior edge 760 of the body 758.

As noted previously herein, the inner portion 756 is configured to secure the second coupler 752 to the second shaft (e.g., the other of the driveshaft 632 and impeller shaft 616). In various embodiments, the inner portion 756 includes an opening 757 formed therein that is configured to receive the second shaft. In various embodiments, the inner portion 756 includes a slot 759 formed therein extending radially from the opening defining a key for the second shaft. In these embodiments, the second shaft includes a radial protrusion that is received in the slot 759. In various embodiments, the inner portion 756 protrudes axially from the body 758 at least in one axial direction, such as towards the first coupler 742 when installed on the second shaft.

The second coupler 752 is secured to the second shaft (e.g., the impeller shaft 616 of FIG. 6) to inhibit and/or prevent relative rotational movement therebetween. In various embodiments, the inner portion 756 is joined to the second shaft using biocompatible material adhesives (e.g., glue, epoxy), using biocompatible material fasteners (e.g., bolts, screws, pins, nails), and/or by fusing (e.g., welding, brazing) the second coupler 752 to the second shaft.

The magnets 744 and 754 are sized, shaped, and positioned to ensure a magnetic coupling between the first coupler 742 and the second coupler 752, such that the first coupler 742 and the second coupler 752 rotate together. In various embodiments, the magnets 744 are positioned with rotational symmetry on the body 748, and the magnets 754 are positioned with rotational symmetry on the body 758. In some embodiments, the magnets 744 of the first coupler 742 are substantially the same size as the magnets 754 of the second coupler 752. In additional embodiments, the magnets 744 of the first coupler 742 are a different size than the magnets 754 of the second coupler 752. Additionally, one or more of the magnets 744 of the first coupler 742 may be different shapes and/or sizes from one another. Similarly, one or more magnets 754 of the second coupler 752 may be different shapes and/or sizes from one another.

While the embodiment illustrated in FIG. 7 includes the body 748 and the body 758 with an ‘X’ shape, as will be discussed in further detail below, in various embodiments, the body 748 and the body 758 include other shapes, such as those illustrated in FIGS. 8A-8D. In some embodiments, the first coupler 742 includes a number of magnets 744 that is the same as a number of magnets 754 of the second coupler 752.

FIGS. 8A-8D are front views of embodiments of components of the coupling of FIG. 6. In FIGS. 8A-8D and the associated description, functionally similar features (e.g., structures, materials) as those described above with reference to FIG. 7 are referred to with similar reference numerals incremented by 100. To avoid repetition, not all features shown in FIGS. 8A-8D are described in detail herein. Rather, unless described otherwise below, a feature in FIG. 7 designated by a reference numeral that is a 100 increment of the reference numeral of a previously described feature will be understood to be substantially similar to the previously described feature. The following description in reference to the couplers 842A-842D, applies to both a first coupler (e.g., the first coupler 642, 742) and also applies to the second coupler (e.g., the second coupler 652, 752) of couplings described herein (e.g., the coupling 640, 740).

Referring collectively to FIGS. 8A-8D, the couplers 842A-842D viewed in an axial direction, include a variety of shapes (e.g., circular, rectangular, triangular). Additionally, the couplers 842A-842D include a body 848 and an inner portion 846 configured to secure the respective coupler 542A-542D to a shaft (driveshaft or impeller shaft).

Referring now to FIG. 8A, in various embodiments, the coupler 842A includes a wheel and spoke shape. The body 848 includes an exterior ring and spokes extending between the exterior ring and the inner portion 846. The spokes define openings 851 between the spokes. In the embodiment illustrated, the coupler 842A includes four magnets 844 proximate the exterior edge 850. However, other numbers of magnets 844 and shapes of magnets 844 are also contemplated, such as a singular annular magnet, multiple annular sectors, and the like.

Referring now to FIGS. 8B-8D, in various embodiments, the body 848 defines blades 853 extending from the inner portion 846 to the exterior edge 850. In these embodiments, one or more magnets 844 is secured to each blade 853. In some of these embodiments, the magnets 844 are secured proximate the exterior edge 850 of the blades 853.

Referring to FIG. 8B, the coupler 842B includes two blades 853 extending about 180 degrees from one another. In various embodiments, each blade 853 including an elliptical shape. In various embodiments, the coupler 842B additionally includes two magnets 844, one magnet 844 secured to each blade 853.

Referring to FIG. 8C, in various embodiments, the coupler 842C includes three blades 853 extending about 120 degrees apart from one another. In various embodiments, each blade 853 includes a triangular/circular sector shape. In various embodiments, the coupler 842C includes three magnets 844, with one magnet 844 secured to each blade 853.

Referring to FIG. 8D, in various embodiments, the coupler 842D includes four blades 853 extending about 90 degrees apart from one another. In various embodiments, each blade 853 includes a rectangular shape. In various embodiments, the coupler842D includes a first set of the magnets 844 comprising four magnets 844, with one magnet 844 secured to each blade 853.

Although illustrated as including a magnet on each blade 853, the component 542B-542D may include more blades 853 than magnets 844, or more magnets 844 than blades 853. For example, one or more of the blades 853 may include multiple magnets 844. Additionally, one or more of the blades 853 may include no magnets 844. Further, while each of the couplers 5842A-842D is illustrated with blades of particular shapes, in various embodiments, the couplers include different combinations of blade numbers and blade shapes.

FIG. 9 is a flowchart of a method 900 of operating a circulatory assist device. In various embodiments, the method includes rotating a driveshaft with a motor positioned within a housing, the driveshaft extending from the motor within the housing at act 902. The method further includes transferring a torque from the motor to an impeller across a seal via a coupling, the seal forming a sealed compartment with the housing and the compartment confining the motor and the driveshaft therein at act 904. In various embodiments, the impeller includes an impeller shaft, the impeller shaft substantially axially aligned with the driveshaft and offset from the driveshaft with the seal extending therebetween, and the coupling configured to magnetically couple the driveshaft to the impeller shaft.

In various embodiments of the method, the coupling includes: a first coupler including a first inner portion configured to be secured to the driveshaft, a first body extending from the inner portion, and first magnets joined to the body; and a second coupler offset from and separate from the first coupler with a gap therebetween, the second coupler including a second inner portion configured to be secured to the impeller shaft, a second body extending from the second inner portion, and second magnets joined to the body, and act 904 includes magnetically coupling the first magnets to the second magnets. In some of these various embodiments, the first magnets are in a position relative to the second magnets chosen from (1) axially offset and radially aligned, (2) axially aligned and radially offset, and (3) at least one first magnet axially offset and radially aligned relative to at least one second magnet and at least another first magnet axially aligned and radially offset from at least another second magnet. In some of these various embodiments, the first inner portion includes a first opening and a first slot extending from the first opening formed therein with the driveshaft received in the first opening, and the second inner portion includes a second opening and a second slot extending from the second opening formed therein with the impeller shaft received in the second opening, and act 904 includes transferring the torque from the driveshaft to the first coupler, transferring the torque from the first coupler to the second coupler magnetically via the first magnets and the second magnets, and transferring the torque from the second coupler to the impeller shaft.

In various embodiments of the method, the seal forms a hermetic seal with the housing. In various embodiments, the housing includes an opening with a radial bearing positioned therein, the radial bearing positioned between the housing and the impeller shaft, the impeller shaft extending through the opening.

In the Brief Summary above and in the Detailed Description, the claims below, and in the accompanying drawings, reference is made to particular features (including method acts) of the disclosure. It is to be understood that the disclosure includes all possible feasible combinations of such particular features. For example, where a particular feature is disclosed in the context of a particular embodiment, or a particular claim, that feature can also be used, to the extent possible, in combination with and/or in the context of other particular aspects and embodiments described herein.

The following description provides specific details, such as components, assembly, and materials in order to provide a thorough description of embodiments of the disclosure. However, a person of ordinary skill in the art will understand that the embodiments of the disclosure may be practiced without employing these specific details.

The use of the term “for example,” means that the related description is explanatory, and though the scope of the disclosure is intended to encompass the examples and legal equivalents, the use of such terms is not intended to limit the scope of an embodiment or this disclosure to the specified components, acts, features, functions, or the like.

Drawings presented herein are for illustrative purposes only, and are not meant to be actual views of any particular material, component, structure, or device. Thus, embodiments described herein are not to be construed as being limited to the particular shapes or regions as illustrated, but include deviations in shapes that result, for example, from manufacturing. For example, a region illustrated or described as box-shaped may have rough and/or nonlinear features, and a region illustrated or described as round may include some rough and/or linear features. Moreover, sharp angles that are illustrated may be rounded, and vice versa. Thus, the regions illustrated in the figures are schematic in nature, and their shapes are not intended to illustrate the precise shape of a region and do not limit the scope of the present claims. The drawings are not necessarily to scale. Additionally, elements common between figures may retain the same numerical designation.

As used herein, the term “configured” refers to a size, shape, material composition, material distribution, orientation, and arrangement of one or more of at least one structure and at least one apparatus facilitating operation of one or more of the structure and the apparatus in a predetermined way.

As used herein, the terms “comprising” and “including,” and grammatical equivalents thereof include both open-ended terms that do not exclude additional, unrecited elements or method acts, and more restrictive terms such as “consisting of” and “consisting essentially of” and grammatical equivalents thereof.

As used herein, the term “may” with respect to a material, structure, feature, or method act indicates that such is contemplated for use in implementation of an embodiment of the disclosure and such term is used in preference to the more restrictive term “is” so as to avoid any implication that other, compatible materials, structures, features and methods usable in combination therewith should or must be, excluded.

As used herein, the singular forms “a,” “an,” and “the” include the plural forms as well, unless the context clearly indicates otherwise.

As used herein, the term “and/or” includes any and all combinations of one or more of the associated listed items.

As used herein, relational terms, such as “first,” “second,” etc., are used for clarity and convenience in understanding the disclosure and accompanying drawings and does not connote or depend on any specific preference, orientation, or order, except where the context clearly indicates otherwise.

As used herein, the term “about,” when used in reference to a numerical value for a particular parameter, is inclusive of the numerical value and a degree of variance from the numerical value that one of ordinary skill in the art would understand is within acceptable tolerances for the particular parameter. For example, “about,” in reference to a numerical value, may include additional numerical values within a range of from 90.0 percent to 110.0 percent of the numerical value, such as within a range of from 95.0 percent to 105.0 percent of the numerical value, within a range of from 97.5 percent to 102.5 percent of the numerical value, within a range of from 99.0 percent to 101.0 percent of the numerical value, within a range of from 99.5 percent to 100.5 percent of the numerical value, or within a range of from 99.9 percent to 100.1 percent of the numerical value.

As used herein, the term “substantially” in reference to a given parameter, property, or condition means and includes to a degree that one of ordinary skill in the art would understand that the given parameter, property, or condition is met with a degree of variance, such as within acceptable tolerances. By way of example, depending on the particular parameter, property, or condition that is substantially met, the parameter, property, or condition may be at least 90.0 percent met, at least 95.0 percent met, at least 99.0 percent met, at least 99.9 percent met, or even 100.0 percent met.

As used herein, the terms “biocompatible material” and “biocompatible materials” refer to any materials suitable for being within a subject’s (e.g., a mammal’s, such as a human’s) body. Biocompatible materials include ceramics and ceramic composites such as alumina, zirconia, hydroxyapatite, and bioglass (e.g., composites including silica (SiO2), calcium, sodium oxide, hydrogen, and/or phosphorous). As non-limiting examples, bioglass may include 45S5 (e.g., 45% SiO₂, 24.5% CaO, 24.5% Na₂O, and 6% (P₂O₅)) and additional compositions described in the following article, the contents of which are incorporated herein by this reference: Bioglass: A novel biocompatible innovation, Vidya Krishnan and T. Lakshmi, Journal of Advanced Pharmaceutical Technology & Research, Volume 4(2), Apr-June 2013, pages 78-83, DOI: 10.4103/2231-4040.111523. Biocompatible materials may additionally include metals and metal alloys, such as stainless steel, titanium and titanium alloys (e.g., Nitinol), cobalt-chromium alloys (e.g., ASTM F75). Furthermore, biocompatible material may include polymers, such as polyvinylchloride (PVC), polyethylene (PE), polypropylene (PP), polytetrafluoroethylene (PTFE), polymethylmethacrylate (PMMA), trimethylcarbonate (C₄H₆O₃), TMC NAD-lactide (CH₃[C₆H₈O₄]m[C₄H₆O₃]nCH₃), polylactic acid (PLA), and medical-grade silicone.

The embodiments of the disclosure described above and illustrated in the accompanying drawings do not limit the scope of the disclosure, which is encompassed by the scope of the appended claims and their legal equivalents. Any equivalent embodiments are within the scope of this disclosure. Indeed, various modifications of the disclosure, in addition to those shown and described herein, such as alternative useful combinations of the elements described, will become apparent to those skilled in the art from the description. Such modifications and embodiments also fall within the scope of the appended claims and equivalents.

What is claimed is:

1. A coupling for a circulatory assist device, the coupling comprising:
a first coupler including a first inner portion configured to be secured to a first shaft, a first body extending from the inner portion, and first magnets joined to the body; and
a second coupler offset from and separate from the first coupler with a gap therebetween, the second coupler including a second inner portion configured to be secured to a second shaft, a second body extending from the second inner portion, and second magnets joined to the body, the second magnets magnetically coupled to the first magnets and configured to transfer a torque applied to one of the first shaft and the second shaft to an other of the first shaft and the second shaft.

2. The coupling of claim 1, wherein the gap is configured to receive a seal therein, the seal being spaced apart from the first coupler and the second coupler.

3. The coupling of claim 1, wherein the first magnets are in a position relative to the second magnets chosen from (1) axially offset and radially aligned, (2) axially aligned and radially offset, and (3) at least one first magnet axially offset and radially aligned relative to at least one second magnet and at least another first magnet axially aligned and radially offset from at least another second magnet.

4. The coupling of claim 1, wherein the first inner portion includes a first opening and a first slot extending from the first opening formed therein and configured to receive the first shaft, and the second inner portion includes a second opening and a second slot extending from the second opening formed therein and configured to receive the second shaft.

5. The coupling of claim 1, wherein the first magnets are symmetrically positioned on the first body and the second magnets are symmetrically positioned on the second body to align with the first magnets.

6. The coupling of claim 1, wherein the first body includes multiple first blades extending from the first inner portion and the first magnets include at least one first magnet secured to each of the multiple first blades, and wherein the second body includes multiple second blades extending from the second inner portion and the second magnets include at least one second magnet secured to each of the multiple second blades.

7. The coupling of claim 1, wherein the second coupler is configured to contact bodily fluids of a subject and the second magnets include neodymium or samarium-cobalt.

8. A circulatory assist device comprising:
a motor positioned within a housing;
a driveshaft extending from the motor within the housing;
a seal forming a sealed compartment with the housing, the compartment confining the motor, and the driveshaft therein;
an impeller including an impeller shaft, the impeller shaft substantially axially aligned with the driveshaft and offset from the driveshaft with the seal extending therebetween; and
a coupling configured to magnetically couple the driveshaft to the impeller shaft and to transfer torque from the motor to the impeller across the seal.

9. The circulatory assist device of claim 8, wherein the coupling includes:
a first coupler including a first inner portion configured to be secured to the driveshaft, a first body extending from the inner portion, and first magnets joined to the body; and
a second coupler offset from and separate from the first coupler with a gap therebetween, the second coupler including a second inner portion configured to be secured to the impeller shaft, a second body extending from the second inner portion, and second magnets joined to the body, the second magnets magnetically coupled to the first magnets and configured to transfer the torque.

10. The circulatory assist device of claim 9, wherein the first magnets are in a position relative to the second magnets chosen from (a) axially offset and radially aligned, (b) axially aligned and radially offset, and (c) at least one first magnet axially offset and radially aligned relative to at least one second magnet and at least another first magnet axially aligned and radially offset from at least another second magnet.

11. The circulatory assist device of claim 9, wherein the first inner portion includes a first opening and a first slot extending from the first opening formed therein with the driveshaft received in the first opening, and the second inner portion includes a second opening and a second slot extending from the second opening formed therein with the impeller shaft received in the second opening.

12. The circulatory assist device of claim 9, wherein the first magnets are symmetrically positioned on the first body and the second magnets are symmetrically positioned on the second body to align with the first magnets.

13. The circulatory assist device of claim 8, wherein the seal forms a hermetic seal with the housing.

14. The circulatory assist device of claim 8, wherein the housing includes an opening with a radial bearing positioned therein, the radial bearing positioned between the housing and the impeller shaft, the impeller shaft extending through the opening.

15. A method of operating a circulatory assist device, comprising:
rotating a driveshaft with a motor positioned within a housing, the driveshaft extending from the motor within the housing;
transferring a torque from the motor to an impeller across a seal via a coupling, the seal forming a sealed compartment with the housing and the compartment confining the motor and the driveshaft therein,
wherein the impeller includes an impeller shaft, the impeller shaft substantially axially aligned with the driveshaft and offset from the driveshaft with the seal extending therebetween, and the coupling configured to magnetically couple the driveshaft to the impeller shaft.

16. The method according to claim 15, wherein the coupling includes:
a first coupler including a first inner portion configured to be secured to the driveshaft, a first body extending from the inner portion, and first magnets joined to the body; and
a second coupler offset from and separate from the first coupler with a gap therebetween, the second coupler including a second inner portion configured to be secured to the impeller shaft, a second body extending from the second inner portion, and second magnets joined to the body,
wherein transferring the torque includes magnetically coupling the first magnets to the second magnets.

17. The method according to claim 16, wherein the first magnets are in a position relative to the second magnets chosen from (a) axially offset and radially aligned, (b) axially aligned and radially offset, and (c) at least one first magnet axially offset and radially aligned relative to at least one second magnet and at least another first magnet axially aligned and radially offset from at least another second magnet.

18. The method according to claim 16, wherein the first inner portion includes a first opening and a first slot extending from the first opening formed therein with the driveshaft received in the first opening, and the second inner portion includes a second opening and a second slot extending from the second opening formed therein with the impeller shaft received in the second opening, and wherein transferring the torque includes transferring the torque from the driveshaft to the first coupler, transferring the torque from the first coupler to the second coupler magnetically via the first magnets and the second magnets, and transferring the torque from the second coupler to the impeller shaft.

19. The method according to claim 15, wherein the seal forms a hermetic seal with the housing.

20. The method according to claim 15, wherein the housing includes an opening with a radial bearing positioned therein, the radial bearing positioned between the housing and the impeller shaft, the impeller shaft extending through the opening.
